(19) United States
(12) Patent Application Publication
(10) Pub. No.: US 2020/0171063 A1
(43) Pub. Date: Jun. 4, 2020

(54) Use Of Cholesterol For Promoting Survival And Proliferation Of Primary Medulloblastoma Cells

(71) Applicant: Institute For Cancer Research d/b/a The Research Institute Of Fox Chase Cancer Center, Philadelphia, PA (US)

(72) Inventors: Zeng-Jie Yang, Philadelphia, PA (US); Renata E. Gordon, Philadelphia, PA (US)

(21) Appl. No.: 16/616,055

(22) PCT Filed: May 25, 2018
(86) PCT No.: PCT/US2018/034581

§ 371 Date: Nov. 22, 2019

Related U.S. Application Data
(60) Provisional application No. 62/511,370, filed on May 26, 2017.

(51) Int. Cl. A61K 31/7048; A61K 31/4402; A61K 31/58; A61K 31/5377; A61K 31/506; A61K 31/496; A61K 31/454; A61K 31/497; A61K 31/155; A61K 31/4704; A61K 31/4184; A61K 31/517; C12N 5/00

(52) U.S. Cl. CPC A61K 31/7048; A61K 31/4402; A61K 31/58; A61K 31/5377; A61K 31/506; A61K 31/496; A61K 45/06; A61K 31/497; A61K 31/155; A61K 31/4704; A61K 31/4184; A61K 31/517; C12N 5/0018; A61K 31/454

(57) ABSTRACT

Methods of treatment of sonic hedgehog associated malignancies in addition to methods that support the survival and proliferation of medulloblastoma cells in culture are provided.

### FIELD

The present disclosure is directed, in part, to methods of treatment of hedgehog pathway associated malignancies.

### BACKGROUND

Cholesterol is a key component of the cellular membrane and a precursor of steroid hormones and bile acid (Simons et al., Science, 2000, 290, 1721-1726). Besides being an important constituent of the brain cell membrane, cholesterol is a pivotal signaling molecule for brain morphology and function (Orth et al., Cholesterol, 2012, 292, 598). Extensive studies have reported that cholesterol plays an important role in the regulation of hedgehog (Hh) signaling pathway (Riobo, Curr. Opin, Pharmacology, 2012, 12, 736-741). Hh signaling pathway is essential for normal vertebrate development in that it regulates cell proliferation and differentiation in a variety of tissues. In the absence of the Hh ligand, the antagonizing receptor, Patched1 (Ptch1) inhibits the downstream transducers of Hh pathway by tethering the seven-transmembrane protein, Smoothened (Smo), which prevents the downstream activation in Hh signaling cascade. When Hh ligand is present, the interaction between Hh and Ptch1 relieves the inhibition of Smo, which then triggers a cascade of downstream events culminating in the activation of glioma-associated oncogenes (Gli). It has been demonstrated that cholesterol is required for post-transcriptional modification of Hh ligand, which appears to be necessary for the generation and diffusion of functional Hh ligand (Bhatia et al., Acta Neuropathologica, 2012, 123, 587-600).

Aberrant activation of the Hh signal pathway promotes the formation of medulloblastoma (MB), the most common malignant brain tumor in children (Yang et al., Cancer Cell, 2008, 14, 135-145). Despite the advance in the understanding of MB, a significant proportion of patients still succumb to this disease. In addition, the conventional MB treatment, including surgery, chemotherapy and radiation, causes severe long-term side effects including cognitive deficits and endocrine disorders (Klesse et al., CNS Drugs, 2010, 24, 285-301, and Roussel et al., F1000 Biol. Rep., 2011, 3, 5). Therefore, improved strategies for treating MB are highly needed.

The present application demonstrates that enhanced cholesterol biosynthesis in Hh subtype MB promotes tumor cell proliferation and in vivo tumor growth. In addition, an antagonist of cholesterol biosynthesis, simvastatin exhibited beneficial effects in MB treatment. Further, simvastatin synergized with the conventional Smo antagonist, vismodegib (GDC0449), in prohibiting MB progression in vivo.

### SUMMARY

The present disclosure provides pharmaceutical compositions comprising a cholesterol synthesis inhibitor, an antagonist of the transmembrane protein Smoothened (Smo), and a pharmaceutically acceptable carrier.

The present disclosure also provides methods of reducing the proliferation of Hh-associated tumor cell or treating an Hh-associated cancer in a human in need thereof comprising administering to the human a cholesterol synthesis inhibitor. In some embodiments, the human is also administered a Smo antagonist.

The present disclosure also provides pharmaceutical compositions comprising a cholesterol synthesis inhibitor for use in the manufacture of a medicament for reducing the proliferation of an Hh-associated tumor cell or treating an Hh-associated cancer in a human.

The present disclosure also provides uses of pharmaceutical composition comprising a cholesterol synthesis inhibitor for reducing the proliferation of an Hh-associated tumor cell or treating an Hh-associated cancer in a human.

The present disclosure also provides uses of any one or more of the pharmaceutical compositions described herein for reducing the proliferation of an Hh-associated tumor cell or treating an Hh-associated cancer in a human.

The present disclosure also provides methods of culturing medulloblastoma cells comprising contacting the medulloblastoma cells with growth media, and contacting the medulloblastoma cells with an amount of cholesterol that is sufficient to maintain high cellular proliferation in culture for up to 96 hours at least.

The present disclosure also provides kits comprising growth media for culturing medulloblastoma cells, and cholesterol.

### BRIEF DESCRIPTION OF THE DRAWINGS

FIGS. 1A, 1B, 1C, 1D, 1E, 1F, and 1G show Hh type MB exhibited an enhanced cholesterol biosynthesis signature.

FIGS. 2A (panels A, B, C, D, E, F, G, H, I, and J), 2B, and 2C (panels A, B, C, and D) show cholesterol biosynthesis inhibitors blocked MB proliferation in vitro.

FIG. 3 (panels A, B, C, D, E, F, G, H, I, I′, I″, J, J′, J″, K, K′, K″, and L) shows depletion of NAD(P)-dependent steroid dehydrogenase-like (NSDHL) abolished Hh-dependent MB cell proliferation.

FIG. 4 (panels A, B, C, D, E, and F) shows cholesterol promotes Smo activation during Hh signal transduction.

FIG. 5 (panels A, B, C, D, D′, D″, E, E′, E″, F, F′, F″, G, and H) shows simvastatin blocked MB allograft growth and synergized with Smo antagonist.

### DESCRIPTION OF EMBODIMENTS

Unless defined otherwise, all technical and scientific terms have the same meaning as is commonly understood by one of ordinary skill in the art to which the embodiments disclosed belongs.

As used herein, the terms “a” or “an” means that “at least one” of “one or more” unless the context clearly indicates otherwise.

As used herein, the term “about” means that the numerical value is approximate and small variations would not significantly affect the practice of the disclosed embodiments. Where a numerical value can vary ±10% and remain with the scope of the disclosed embodiments.

As used herein, the terms “antagonize” and “antagonizing” mean reducing or completely eliminating one or more effects.

As used herein, the term “carrier” means a diluent, adjuvant or excipient with which a compound is administered. Pharmaceutical carriers can be liquids, such as water and oils, including those of petroleum, animal, vegetable or synthetic origin, such as peanut oil, soybean oil, mineral oil, sesame oil and the like. The pharmaceutical carriers can also be saline, gum, acacia, gelatin, starch paste, talc, keratin, colloidal silica, urea and the like. In addition, auxiliary, stabilizing, thickening, lubricating and coloring agents can be used.

As used herein, the terms “comprising” (and any form of comprising, such as “comprise”, “comprises” and “comprised”), “having” (and any form of having, such as “have” and “has”), “including” (and any form of including, such as “contains” and “contain”), are inclusive or open-ended and do not exclude additional, unrecited elements of method steps.

As used herein, the term “contacting” means bringing together one or more compounds with a particular cell, tissue, or organ, in an in vitro system or an in vivo system.

As used herein, the term “individual” or “patient,” used interchangeably, means any animal, including mammals, such as humans.

As used herein, the phrase “in need thereof” means that the animal or mammal has been identified as having a need for the particular method or treatment. In some embodiments, the identification can be by any means of diagnosis. In any of the methods and treatments described herein, the animal or mammal can be in need thereof.

As used herein, the phrase “pharmaceutically acceptable” means those compounds, materials, compositions, and/or dosage forms which are, within the scope of sound medical judgment, suitable for use in contact with tissues of humans and animals. In some embodiments, “pharmaceutically acceptable” means approved by a regulatory agency of the Federal of a state government or listed in the U.S. Pharmacopeia or other generally recognized pharmacopeia for use in animals, and more particularly in humans.

As used herein, the phrase “pharmaceutically acceptable salt(s),” includes, but is not limited to, salts of acidic or basic groups. Compounds that are basic in nature are capable of forming a wide variety of salts with various inorganic and organic acids. Acids that may be used to prepare pharmaceutically acceptable acid addition salts of such basic compounds are those that form non-toxic acid addition salts, i.e., salts containing pharmacologically acceptable anions including, but not limited to, sulfuric, thiosulfuric, citric, maleic, acetic, oxalic, hydrochloride, hydrobromide, hydroiodide, nitrate, sulfate, bisulfate, bisulfite, phosphate, acid phosphate, isonicotinate, borate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, bicarbonate, malonate, mesylate, esylate, napsydisylate, tosylate, besylate, orthophoshate, trifluoroacetate, and pamoate (i.e., 1,1′-methylene-bis-(2-hydroxy-3-naphthoate)) salts. Compounds that include an amino moiety may form pharmaceutically acceptable salts with various amino acids, in addition to the acids mentioned above. Compounds that are acidic in nature are capable of forming base salts with various pharmacologically acceptable cations. Examples of such salts include, but are not limited to, alkali metal or alkaline earth metal salts and, particularly, calcium, magnesium, ammonium, sodium, lithium, zinc, potassium, and iron salts. Salts also includes quaternary ammonium salts of the compounds described herein, where the compounds have one or more tertiary amine moiety.

As used herein, the terms “treat,” “treated,” or “treating” mean therapeutic treatment wherein the object is to prevent or slow down (lessen) an undesired physiological condition, disorder or disease, or obtain beneficial or desired clinical results. For purposes herein, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment includes eliciting a clinically significant response, optionally without excessive levels of side effects. Treatment also includes prolonging survival as compared to expected survival if not receiving treatment.

The present disclosure provides pharmaceutical compositions comprising a cholesterol synthesis inhibitor, an antagonist of the transmembrane protein Smoothened (Smo), and a pharmaceutically acceptable carrier.

In some embodiments, the cholesterol synthesis inhibitor is a Sonic hedgehog (Hh) antagonist, a PCSK9 inhibitor, a cholesterol absorption inhibitor, or a statin. In some embodiments, the statin is simvastatin, atorvastatin, fluvastatin, lovastatin, pravastatin, cerivastatin, mevastatin, pitavastatin, or rosuvastatin. In some embodiments, the statin is simvastatin, atorvastatin, fluvastatin, lovastatin, pravastatin, or rosuvastatin. In some embodiments, the statin is simvastatin.

In some embodiments, the cholesterol synthesis inhibitor is present in amount from about 0.1 mg to about 250 mg, from about 1 mg to about 100 mg, from about 5 mg to about 50 mg, from about 7.5 mg to about 40 mg, or from about 10 mg to about 30 mg. In some embodiments, the cholesterol synthesis inhibitor is present in amount from about 5 mg to about 100 mg, from about 5 mg to about 90 mg, from about 5 mg to about 80 mg, from about 5 mg to about 70 mg, from about 5 mg to about 60 mg, from about 5 mg to about 50 mg, from about 5 mg to about 40 mg, from about 5 mg to about 30 mg, from about 5 mg to about 20 mg, or from about 5 mg to about 10 mg. In some embodiments, the cholesterol synthesis inhibitor is present in amount from about 5 mg to about 50 mg, from about 1 mg to about 50 mg, from about 5 mg to about 50 mg, from about 10 mg to about 50 mg, from about 15 mg to about 50 mg, from about 20 mg to about 50 mg, from about 25 mg to about 50 mg, from about 30 mg to about 50 mg, from about 35 mg to about 50 mg, from about 40 mg to about 50 mg, or from about 45 mg to about 50 mg.

In some embodiments, the Smo antagonist is vismodegib (GDC0449), cyclopamine, erismodegib, PF-5274857, GANT61, SANT1, glasdegib, taladegib, sonidegib, IPI-926, PF-04449913, Ly-2940680, MRT-83, GSA-10, tomatidine, SANT-2, or BMS-833923. In some embodiments, the Smo antagonist is vismodegib (GDC0449).

In some embodiments, the Smo antagonist is present in an amount from about 0.1 mg to about 600 mg, from about 5 mg to about 500 mg, from about 15 mg to about 400 mg, from about 25 mg to about 300 mg, from about 40 mg to about 250 mg, or from about 50 mg to about 150 mg. In some embodiments, the Smo antagonist is present in an amount from about 0.1 mg to about 300 mg, from about 5 mg to about 300 mg, from about 10 mg to about 300 mg, from about 20 mg to about 300 mg, from about 30 mg to about 300 mg, from about 40 mg to about 300 mg, from about 50 mg to about 300 mg, from about 60 mg to about 300 mg, from about 80 mg to about 300 mg, from about 100 mg to about 300 mg, from about 200 mg to about 300 mg. In some embodiments, the Smo antagonist is present in an amount from about 5 mg to about 600 mg, from about 5 mg to about 500 mg, from about 5 mg to about 400 mg, from about 5 mg to about 300 mg, from about 5 mg to about 200 mg, from about 5 mg to about 100 mg, or from about 5 mg to about 50 mg.

In some embodiments, the Smo antagonist is vismodegib and the cholesterol synthesis inhibitor is simvastatin. In some embodiments, the ratio of the cholesterol synthesis inhibitor to the Smo antagonist is from about 0.001:1 to about 1000:1 w/w, from 0.005:1 to about 500:1 w/w, from 0.01:1 to about 100:1 w/w, from 0.1:1 to about 10:1 w/w, or from 0.5:1 to about 5:1 w/w. In some embodiments, the ratio of the cholesterol synthesis inhibitor to the Smo antagonist is from about 0.01:1 to about 100:1 w/w. In some embodiments, the cholesterol synthesis inhibitor is present in amount from about 5 mg to about 50 mg, and the Smo antagonist is present in an amount from about 25 mg to about 300 mg.

In some embodiments, the pharmaceutical composition is an oral dosage formulation, an intravenous dosage formulation, a topical dosage formulation, an intraperitoneal dosage formulation, or an intrathecal dosage form.

In some embodiments, the pharmaceutical composition is an oral dosage formulation in the form of a pill, tablet, capsule, cachet, gel-cap, pellet, powder, granule, or liquid.

In some embodiments, the pharmaceutical composition the oral dosage formulation is protected from light and present within a blister pack, bottle, or intravenous bag.

The compounds and compositions described herein can be formulated for parenteral administration by injection, such as by bolus injection or continuous infusion. The compounds and compositions can be administered by continuous infusion subcutaneously over a period of about 15 minutes to about 24 hours. The compositions can take such forms as suspensions, solutions or emulsions in oily or aqueous vehicles, and can contain formulary agents such as suspending, stabilizing and/or dispersing agents. In some embodiments, the injectable is in the form of short-acting, depot, or implant and pellet forms injected subcutaneously or intramuscularly. In some embodiments, the parenteral dosage form is the form of a solution, suspension, emulsion, or dry powder.

For oral administration, the compounds and compositions described herein can be formulated by combining the compounds with pharmaceutically acceptable carriers well known in the art. Such carriers enable the compounds to be formulated as tablets, pills, dragees, capsules, emulsions, liquids, gels, syrups, caches, pellets, powders, granules, slurries, lozenges, aqueous or oily suspensions, and the like, for oral ingestion by a patient to be treated. Pharmaceutical preparations for oral use can be obtained by, for example, adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations including, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, including, but not limited to, the cross-linked polyvinyl pyrrolidone, agar, or alginic acid or a salt thereof such as sodium alginate.

Orally administered compositions can contain one or more optional agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, when in tablet or pill form, the compositions may be coated to delay disintegration and absorption in the gastrointestinal tract thereby providing a sustained action over an extended period of time. Selectively permeable membranes surrounding an osmotically active driving compound are also suitable for orally administered compounds. Oral compositions can include standard vehicles such as, for example, mannitol, lactose, starch, magnesium stearate, sodium saccharine, cellulose, magnesium carbonate, etc. Such vehicles are suitably of pharmaceutical grade.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added.

For buccal administration, the compositions can take the form of, such as, tablets or lozenges formulated in a conventional manner.

For administration by inhalation, the compounds and compositions described herein can be delivered in the form of an aerosol spray presentation from pressurized packs or a nebulizer, with the use of a suitable propellant, such as dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, such as gelatin for use in an inhaler or insufflator can be formulated containing a powder mix of the compound and a suitable powder base such as lactose or starch.

The compounds and compositions described herein can also be formulated in rectal compositions such as suppositories or retention enemas, such as containing conventional suppository bases such as cocoa butter or other glycerides. The compounds and compositions described herein can also be formulated in vaginal compositions such as vaginal creams, suppositories, pessaries, vaginal rings, and intrauterine devices.

In transdermal administration, the compounds and compositions can be applied to a plaster, or can be applied by transdermal, therapeutic systems that are consequently supplied to the organism. In some embodiments, the compounds and compositions are present in creams, solutions, powders, fluid emulsions, fluid suspensions, semi-solids, ointments, pastes, gels, jellies, and foams, or in patches containing any of the same.

The compounds and compositions described herein can also be formulated as a depot preparation. Such long acting formulations can be administered by implantation (for example subcutaneously or intramuscularly) or by intramuscular injection. Depot injections can be administered at about 1 to about 6 months or longer intervals. Thus, for example, the compounds and compositions can be formulated with suitable polymeric or hydrophobic materials (for example as an emulsion in an acceptable oil) or ion exchange resins, or as sparingly soluble derivatives, for example, as a sparingly soluble salt.

In some embodiments, the compounds and compositions can be delivered in a controlled release system. In some embodiments, a pump may be used (see Langer, supra; Sefton, CRC Crit. Ref. Biomed. Eng., 1987, 14, 201; Buchwald et al., Surgery, 1980, 88, 507 Saudek et al., N. Engl. J. Med., 1989, 321, 574). In some embodiments, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger et al., J. Macromol. Sci. Rev. Macromol. Chem., 1983, 23, 61; see, also Levy et al., Science, 1985, 228, 190; During et al., Ann. Neurol., 1989, 25, 351; Howard et al., J. Neurosurg., 1989, 71, 105). In some embodiments, a controlled-release system can be placed in proximity of the target of the compounds and compositions described herein, such as the liver, thus requiring only a fraction of the systemic dose (see, e.g., Goodson, in Medical Applications of Controlled Release, supra, vol. 2, pp. 115-138 (1984)). Other controlled-release systems discussed in the review by Langer, Science, 1990, 249, 1527-1533) may be used.

The compounds and compositions described herein can be contained in formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The pharmaceutical compositions can also comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols. In some embodiments, the compounds described herein can be used with agents including, but not limited to, topical analgesics (e.g., lidocaine), barrier devices (e.g., GelClair), or rinses (e.g., Caphosol). Pharmaceutical carriers can be liquids, such as water and oils, including those of petroleum, animal, vegetable or synthetic origin, such as peanut oil, soybean oil, mineral oil, sesame oil, and the like. The pharmaceutical carriers can also be saline, gum acacia, gelatin, starch paste, talc, keratin, colloidal silica, urea, and the like. In addition, auxiliary, stabilizing, thickening, lubricating and coloring agents can be used.

In some embodiments, the compounds and compositions described herein can be delivered in a vesicle, in particular a liposome (see, Langer, Science, 1990, 249, 1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

The amount of compound to be administered may be that amount which is therapeutically effective. The dosage to be administered may depend on the characteristics of the subject being treated, e.g., the particular animal treated, age, weight, health, types of concurrent treatment, if any, and frequency of treatments, and on the nature and extent of the disease, condition, or disorder, and can be easily determined by one skilled in the art (e.g., by the clinician). The selection of the specific dose regimen can be selected or adjusted or titrated by the clinician according to methods known to the clinician to obtain the desired clinical response. In addition, in vitro or in vivo assays may optionally be employed to help identify optimal dosage ranges. The precise dose to be employed in the compositions may also depend on the route of administration, and should be decided according to the judgment of the practitioner and each patient's circumstances.

The present disclosure also provides methods of reducing the proliferation of an Hh-associated tumor cell or treating an Hh-associated cancer in a human in need thereof comprising administering to the human one or more cholesterol synthesis inhibitors. Any of the cholesterol synthesis inhibitors described herein, or any of the compositions containing the same described herein, can be administered. In some embodiments, the methods further comprise administering one or more Smo antagonists to the human In some embodiments, the cholesterol synthesis inhibitor is administered prior to the administration of the Smo antagonist, after the administration of the Smo antagonist, or concurrently with administration of the Smo antagonist. In some embodiments, the cholesterol synthesis inhibitor is administered prior to the administration of the Smo antagonist or after the administration of the Smo antagonist. In some embodiments, the cholesterol synthesis inhibitor is administered concurrently with administration of the Smo antagonist. In some embodiments, the cholesterol synthesis inhibitor and the Smo antagonist are present in the same pharmaceutical composition.

In some embodiments, the human is also administered radiation therapy and/or chemotherapy. In some embodiments, the Hh-associated cancer is breast cancer, stomach cancer, liver cancer, prostate cancer, small-cell lung cancer, pancreatic cancer (PDAC), colon cancer, esophagus cancer, biliary tract cancer, medulloblastoma, basal cell carcinoma, or rhabdomyosarcoma. In some embodiments, the Hh-associated cancer is medulloblastoma. In some embodiments, the medulloblastoma is Wnt subtype, Hh subtype, group 3 subtype, or group 4 subtype.

In some embodiments, the human is a child up to 17 years of age. In some embodiments, the human is an adult at least 18 years of age.

The compounds and compositions described herein can be administered by any route of administration including, but not limited to, oral, sublingual, buccal, rectal, intranasal, inhalation, eye drops, ear drops, epidural, intracerebral, intracerebroventricular, intrathecal, epicutaneous or transdermal, subcutaneous, intradermal, intravenous, intraarterial, intraosseous infusion, intramuscular, intracardiac, intraperitoneal, intravesical infusion, and intravitreal. In some embodiments, the administration is oral, sublingual, buccal, rectal, intranasal, inhalation, eye drops, or ear drops. In some embodiments, the administration is oral, sublingual, buccal, rectal, intranasal, or inhalation. In some embodiments, the administration is epidural, intracerebral, intracerebroventricular, or intrathecal. In some embodiments, the administration is epicutaneous or transdermal, subcutaneous, or intradermal. In some embodiments, the administration is intravenous, intraarterial, intraosseous infusion, intramuscular, intracardiac, intraperitoneal, intravesical infusion, or intravitreal. In some embodiments, the administration is intravenous, intramuscular, or intraperitoneal. The route of administration can depend on the particular disease, disorder, or condition being treated and can be selected or adjusted by the clinician according to methods known to the clinician to obtain desired clinical responses. Methods for administration are known in the art and one skilled in the art can refer to various pharmacologic references for guidance (see, for example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980)).

In some embodiments, it may be desirable to administer one or more compounds, or a pharmaceutically acceptable salt thereof, or compositions comprising the same, to a particular area in need of treatment. This may be achieved, for example, by local infusion (for example, during surgery), topical application (for example, with a wound dressing after surgery), by injection (for example, by depot injection), catheterization, by suppository, or by an implant (for example, where the implant is of a porous, non-porous, or gelatinous material, including membranes, such as silastic membranes, or fibers). Formulations for injection can be presented in unit dosage form, such as in ampoules or in multi-dose containers, with an added preservative.

The present disclosure also provides methods of culturing medulloblastoma cells comprising contacting the medulloblastoma cells with growth media and contacting the medulloblastoma cells with an amount of cholesterol that is sufficient to maintain the cells in active proliferative state in culture for at least 96 hours. In some embodiments, the growth media is NB-B27. In some embodiments, the cholesterol is present at a concentration of about 5 μM to about 15 μM. In some embodiments, the medulloblastoma cells are present at a concentration from about 2×10⁵ cells to about 1×10⁷ cells per well of a microtiter plate. In some embodiments, the medulloblastoma cells are NIH3T3 cells, MEF cells, or cells isolated from Ptc^{+/-} mice.

The present disclosure also provides kits comprising growth media and cholesterol for culturing medulloblastoma cells. In some embodiments, the kit further comprises directions for culturing medulloblastoma cells. In some embodiments, the kit further comprises growth media, such as NB-B27. In some embodiments, the kit further comprises a microtiter plate or tissue culture flask.

The present disclosure also provides pharmaceutical compositions comprising a cholesterol synthesis inhibitor for use in the manufacture of a medicament for reducing the proliferation of an Hh-associated tumor cell or treating an Hh-associated cancer in a human. In some embodiments, the medicament further comprises a Smo antagonist.

The present disclosure also provides uses of pharmaceutical compositions comprising a cholesterol synthesis inhibitor for reducing the proliferation of an Hh-associated tumor cell or treating an Hh-associated cancer in a human. In some embodiments, the pharmaceutical compositions further comprise a Smo antagonist.

In order that the subject matter disclosed herein may be more efficiently understood, examples are provided below. It should be understood that these examples are for illustrative purposes only and are not to be construed as limiting the claimed subject matter in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### EXAMPLES

### Example 1: Hh Subtype MB Cells Exhibit Dysregulated Cholesterol Metabolism

One purpose of the present experiment was to examine pathway alterations among MB subtypes.

To examine pathway alterations among MB subtypes, transcriptomes of 103 primary human MBs were analyzed by Geneset Variation Analysis (GSVA) (Alavi-Majd et al., Gene, 2014, 534, 383-389, and Hanzelmann et al., BMC Bioinformatics, 2013, 14, 7). Over 4900 gene sets available in MsigDB (the molecular signature database) including KEGG (Kyoto encyclopedia of genes and genomes) and Biocarta pathways were used for enrichment analysis. To identify significantly enriched pathways among subtypes, a one-way ANOVA with post-hoc Turkey's HSD (honest significance difference) using the enriched pathway GSVA scores was employed. The pathways with adjusted p-value <0.05 were considered significant.

To examine subtype specific pathway alterations in MB, the Northcott et al. (Northcott et al., J. Clin. Oncol., 2011, 29, 1408-1414) MB gene expression data including the well characterized four subtypes was employed. The raw gene expression data, obtained from GEO (GSE21140), was normalized using RMA (Irizarry et al., Biostatistics, 2003, 4, 249-264). Gene Set Variation Analysis (GSVA) (Hanzelmann et al., BMC Bioinformatics, 2013, doi: 10.1186/1471-2105-14-7) on the RMA normalized expression data was applied to identify pathways that are enriched in a single sample (method arguments: function=‘gsva’; mx.diff='FALSE; verbose=FALSE). Over 4900 gene sets compiled from all gene sets available in MsigDB including KEGG and Biocarta pathways were used for enrichment analysis. To identify pathways that differ significantly among subtypes, Kruskal-Wallis test was used to analyze the enrichment scores resulting from GSVA.

To investigate the association between cholesterol and MB tumorigenesis, the expression of proteins involved in cholesterol homeostasis in mouse MB was examined by immunohistochemistry.

Histology and immunohistochemistry: For histological analyses, mice were perfused with PBS, and the cerebellum was removed and fixed in 10% formalin overnight. Samples were embedded in paraffin, and 5 μm sections were prepared and stained with H&E, ABCA1 (Abcam, ab7360, 1:500), NSDHL (Proteintech, 15111-1-AP, 1:100), and SREBP2 (Abcam, ab30682, 1:100).

Frozen sections were prepared according to standard protocol (Yang et al., 2008, Cancer Cell, 14, 135-145). Briefly, tissue was fixed in 4% paraformaldehyde overnight, cryoprotected in 30% sucrose, embedded in O.C.T. and sectioned for 12 μm thick slides. Samples were permeabilized and blocked with 10% normal goat serum on PBST (PBS, 0.1% Triton-X100) and stained against Ki67 (Abcam, ab15580, 1:1000), NeuN (Abcam, ab104224, 1:200), Caspase 3 (Cell Signaling, 9662, 1:500), BrdU (Sigma-Aldrich, B-8434, 1:500), mCherry (Abcam, ab167453, 1:500), Zic1 (Generous gift from Dr. Sigal, Dana Farber Research Institute, 1:200), S1000 (Sigma-Aldrich, 1:500), and YFP (Life Technologies, 1:500).

Cultured primary MB cells were fixed with 4% PFA on PBS for 15 minutes, permeabilized, and blocked in 10% normal goat serum on PBST (PBS, 0.1% Triton-X100) for 1 hour at room temperature. Prior to BrdU immunostaining, cells were incubated in 4.2 mM MgCl₂/0.15 M NaCl pH 5.0 buffer in the presence of 50 U/mL of DNase I (Sigma-Aldrich) at 37° C. for 30 minutes. Secondary antibodies were applied at dilution 1:500 on 10% normal goat serum on PBST for 2 hours at room temperature. Samples were counterstained with DAPI and mounted with Fluoromount-G.

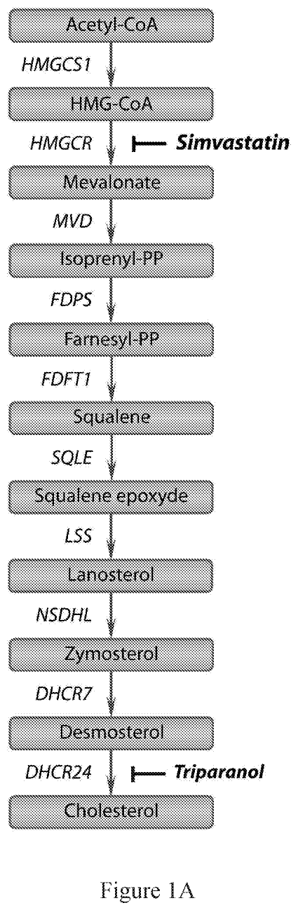

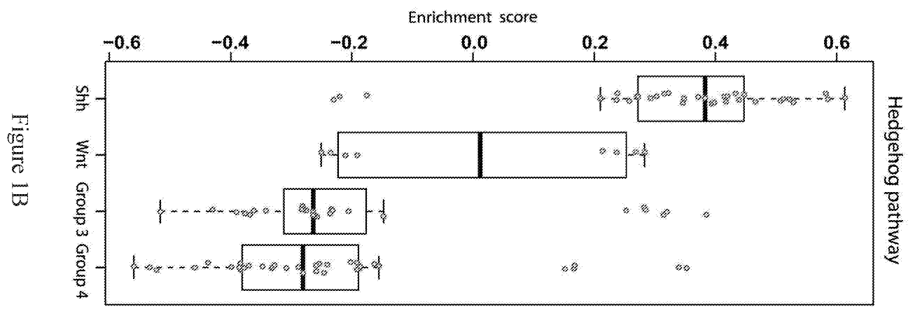

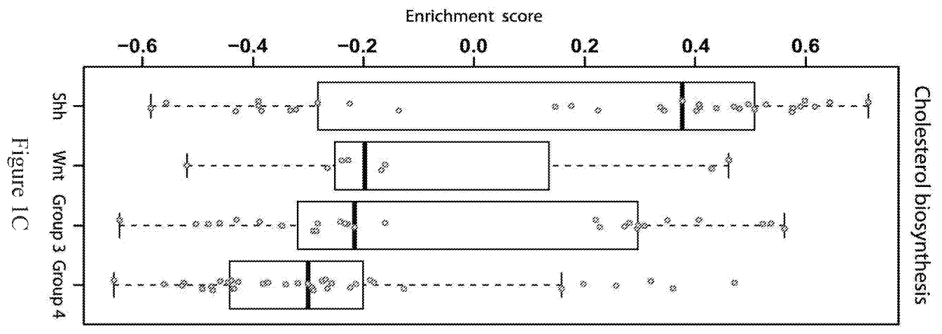

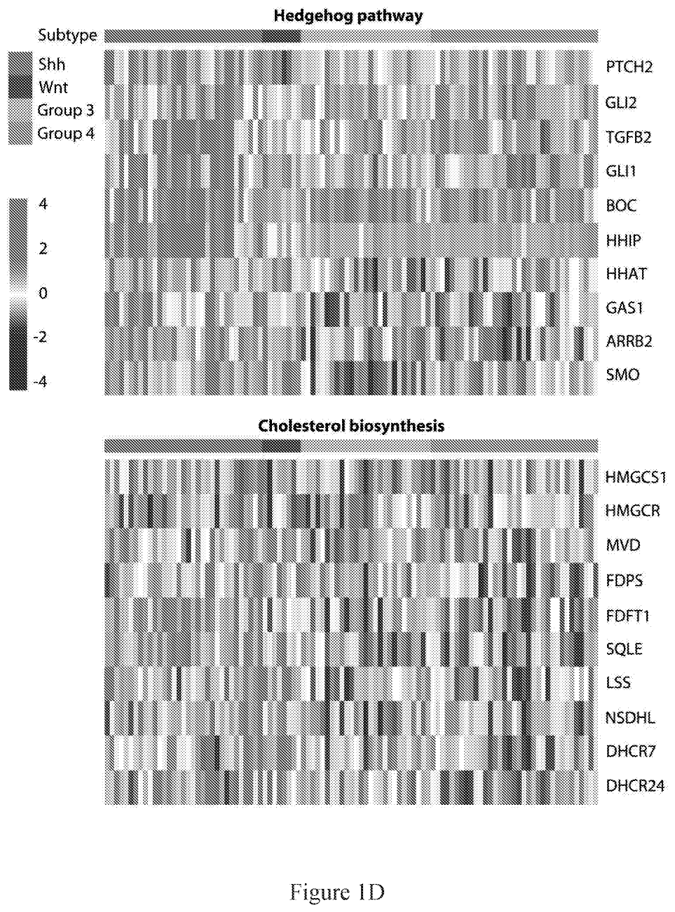

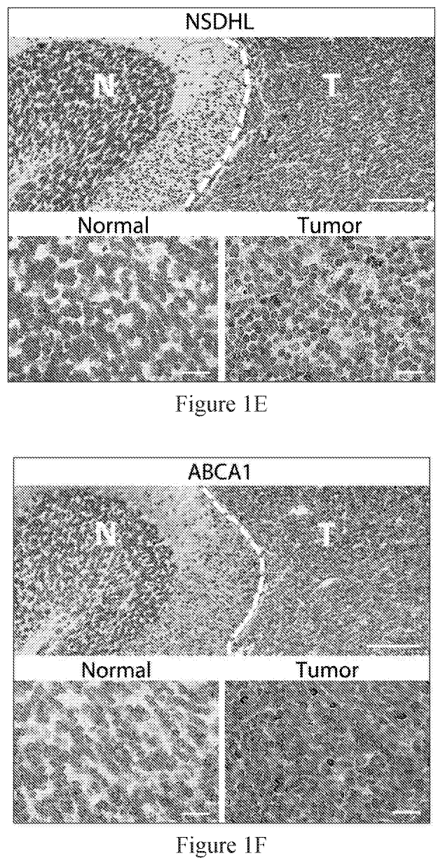

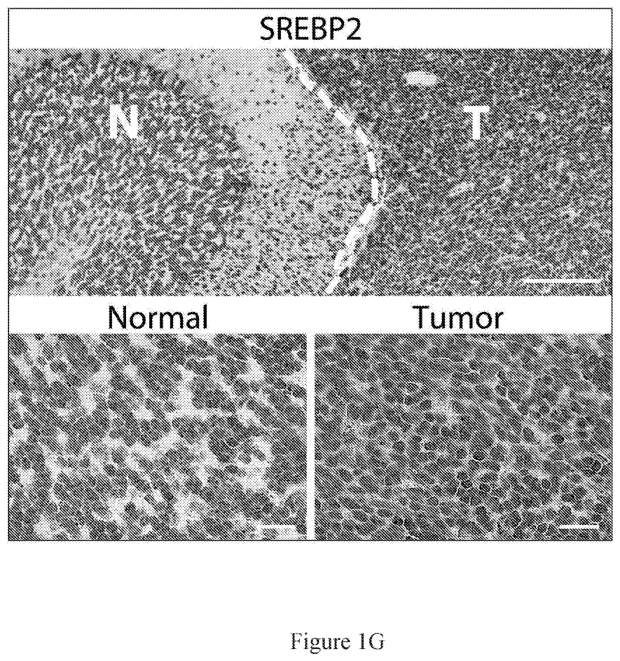

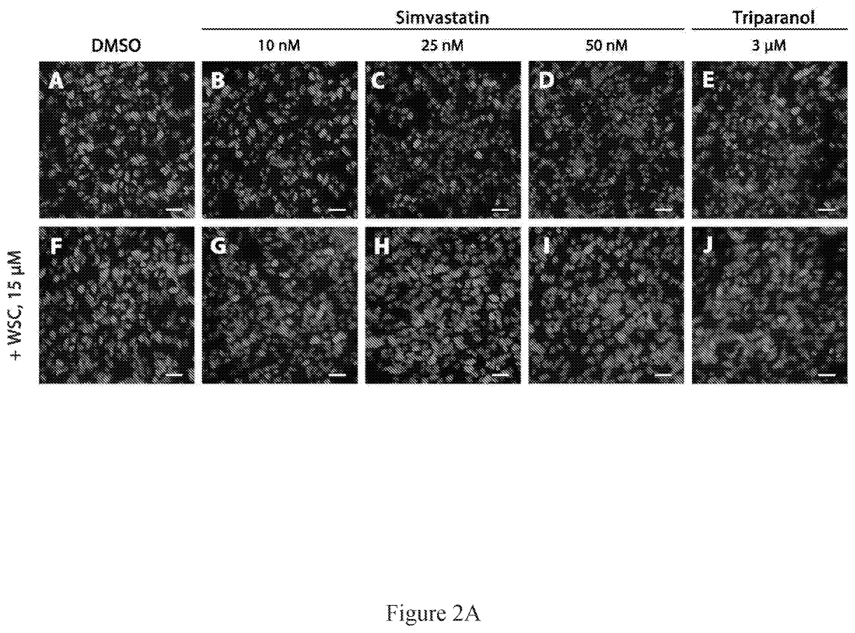

FIG. 1A shows a schematic diagram of the cholesterol biosynthesis pathway with genes encoding corresponding enzymes listed along arrows. Enzymes include: HMGCS1 3-hydroxy-3-methylglutaryl-CoA synthase 1; HMGCR 3-hydroxy-3-methylglutaryl-CoA reductase; MVD mevalonate diphosphate decarboxylase; FDPS farnesyl diphosphate synthase; FDFT1 farnesyl-diphosphate farnesyltransferase 1; SQLE squalene epoxidase; LSS lanosterol synthase; NSDHL NAD(P) dependent steroid dehydrogenase-like; DHCR7 7-dehydrocholesterol reductase; DHCR24 24-dehydrocholesterol reductase. Simvastatin inhibits conversion of HMG-CoA to mevalonate through competitive inhibition of HMG-CoA reductase (HMGCR). Triparanol blocks the latest steps of cholesterol biosynthetic pathway through inhibition of 24-dehydrocholesterol reductase (DHCR24).

FIGS. 1B-1D show gene set enrichment analysis indicating a high cholesterol biosynthesis gene expression pattern among Hh type MB from 103 human samples. In particular, boxplots (see, FIG. 1B and 1C) and heat maps (see, FIG. 1D) show Hh and cholesterol biosynthesis pathway associated gene expression among four subtypes of MB. The above statistical analysis revealed that Hh pathway was up-regulated in Hh type MB compared with other 3 subtypes of MB (see, FIG. 1B). The enrichment score of cholesterol biosynthesis pathway was also overall higher in Hh type MBs (see, FIG. 1C). In addition, expression levels of representative genes in both Hh pathway and cholesterol biosynthesis pathway are remarkably elevated in Hh subtype MB (see, FIG. 1D), supporting that cholesterol biosynthesis may correlate with Hh pathway MB tumorigenesis.

FIGS. 1E-1G show immunohistochemical analysis of NSDHL (see, FIG. 1E), ABCA1 (see, FIG. 1F), and SREBP2 (see, FIG. 1G) in mouse Ptc^{+/-} MB. Normal adjacent tissue represents a cerebellar lobe with differentiated granule neurons used as a control for tumor tissue. Scale bars are 100 μm, insets are 10 μm.

As shown in FIG. 1E, NSDHL was highly expressed in MB tissue, compared with the adjacent cerebellar tissue. The expression of both ABCA1 and SREBP2 proteins was substantially enhanced in tumor tissue, compared with adjacent cerebellum (see, FIGS. 1F and 1G). These results support that cholesterol biosynthesis is elevated in MB tissue.

### Example 2: Cholesterol Maintains In Vitro Proliferation of MB Cells

Whether or not cholesterol is required for proliferation of MB cells in culture was determined.

MB cells isolated from Ptch1^{+/-} mice were cultured with NB-B27 culture medium (lacking cholesterol) in the presence of simvastatin or triparanol at designated concentrations.

Ptch1 ^{+/-} mice were purchased from The Jackson Laboratory.

Cell culture: Primary mouse medulloblastoma cells were isolated from full-blown mouse-derived tumor as described before (Yang et al., 2008, Cancer Cell, 14, 135-145). Obtained cells were cultured on glass coverslips coated with 0.1 mg/ml poly-D-lysine (Millipore, A-003-E). Cells were pulsed with 10 μM of BrdU (Millipore, 19-160) for 2 hours prior to the material collection time point. After being cultured for 48 hours, MB cells were harvested and examined for proliferation by immunocytochemistry.

Histology and immunohistochemistry: Cultured primary MB cells were fixed with 4% PFA on PBS for 15 minutes, permeabilized, and blocked in 10% normal goat serum on PBST (PBS, 0.1% Triton-X100) for 1 hour at room temperature and stained against Ki67 (Abcam, ab15580, 1:1000), NeuN (Abcam, ab104224, 1:200), Caspase 3 (Cell Signaling, 9662, 1:500), BrdU (Sigma-Aldrich, B-8434, 1:500), mCherry (Abcam, ab167453, 1:500), Zic1 (Generous gift from Dr. Sigal, Dana Farber Research Institute, 1:200), S1000 (Sigma-Aldrich, 1:500), YFP (Life Technologies, 1:500).

Prior to BrdU immunostaining, cells were incubated in pH 5.0 buffer for 30 minutes at 37 C. Secondary antibodies were applied at dilution 1:500 on 10% normal goat serum on PBST for 2 hours at room temperature. Samples were counterstained with DAPI and mounted with Fluoromount-G.

Western blotting: Cells and tissues were lysed in RIPA buffer (Thermo) supplemented with protease (Roche) and phosphatase (Thermo) inhibitors cocktail. Total protein lysates were separated in 8% SDS-PAGE and transferred on PVDF membrane. Membranes were probed with antibodies against Gli1 (Cell Signaling, 1:1000) and GAPDH (Sigma-Aldrich, 1:1000). Western Blot signals were detected with SuperSignal West Pico Substrate and exposed on films. To confirm reduced proliferation of MB cells after treatment with simvastatin or triparanol is due to the deficiency of cholesterol biosynthesis, water-soluble cholesterol (WSC) was added into MB culture in the presence of simvastatin or triparanol.

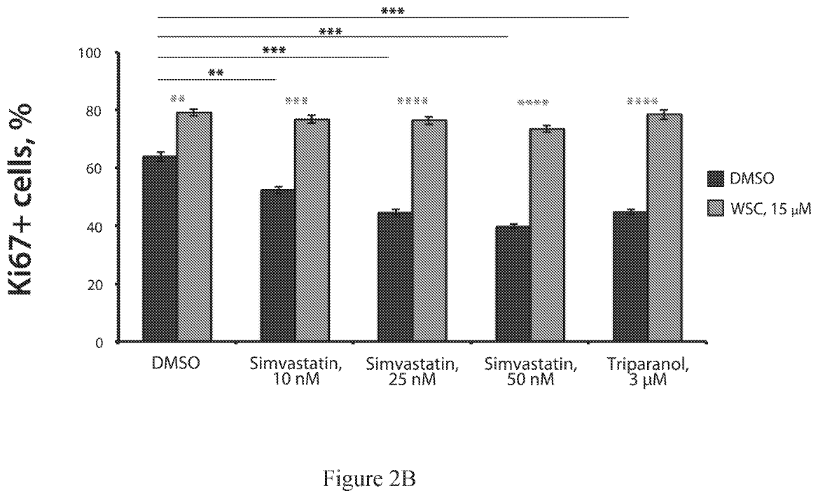

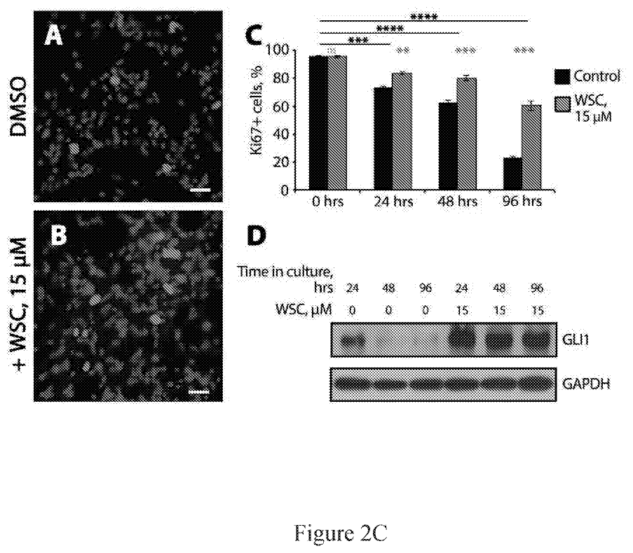

The alterations in MB cell proliferation after inhibition of cholesterol biosynthesis by treatment with simvastatin or triparanol were examined FIG. 2A (panels A-J) show cholesterol biosynthesis inhibitors blocked MB proliferation in vitro. MB cells were isolated from Ptc^{+/-} mouse and cultured in presence of simvastatin (see, FIG. 2A, panels B-D) or triparanol (see, FIG. 2A, panel E), and water-soluble cholesterol (see, FIG. 2A, panels F-J). After 48 hours in vitro, culture cells were fixed, stained against Ki67 (red), and counterstained with DAPI. FIG. 2B shows the percentage of Ki67 positive cells after 48 hours in culture in presence of simvastatin, triparanol and water soluble cholesterol. Significance asterisk key: p value <0.05 (*), p<0.01 (**), p<0.001 (***), p<0.0001 (****).

As shown in FIG. 2A (panel A), about 60% of MB cells were proliferative (Ki67+) in control culture after DMSO treatment. The percentage of proliferating MB cells (Ki67+) in the culture significantly declined as the concentration of simvastatin increased (see, FIG. 2A, panels A-D). Reduced proliferation of MB cells was also observed after treatment with triparanol (see, FIG. 2A, panel E). Repressed proliferation of MB cells after treatment with simvastatin or triparanol was also confirmed by MTT viability assay.

As shown in FIG. 2A (panels F-J), the addition of cholesterol significantly rescued the repressed proliferation of MB cells after simvastatin or triparanol treatment. These data support that cholesterol synthesis is necessary for MB cell proliferation. It should be noted that in the control culture, exogenous cholesterol enhanced MB cell proliferation, which may be due to the insufficient cholesterol biosynthesis in MB cells in vitro.

As shown of FIG. 2C (panels A, B, and C), MB cell proliferation rate in standard culture conditions decreased significantly within 0 to 96 hours time coarse. Addition of 15 μM WSC prolonged tumor cell proliferation, reflected by Ki67 immunocytochemistry. As shown on FIG. 2C (panel D) western blotting analysis of MB cellular lysates under standard condition revealed significant decrease of Gli1 expression. Addition of 15 μM WSC rescued Gli1 expression levels allowing subsequent Hh-driven MB cell proliferation in vitro for 96 hours.

### Example 3: Cholesterol Depletion Inhibits MB Growth

To further investigate the important function of cholesterol in MB tumorigenesis, the effects of genetic blockage of cholesterol synthesis on MB cell proliferation was examined The alterations in MB growth after in vivo blockage of cholesterol synthesis in tumor cells was examined

MB cells isolated from Ptch1^{+/-} mice were infected with a lentivirus carrying mCherry-tagged NSDHL shRNAs or scrambled shRNA.

shRNA lentivirus production: Lentivirus production was performed by utilizing Nsdhl shRNAses and scrambled control shRNA (Genocopiea, MSH029806), pMD2.G and psPAX2 (Addgene) plasmids in accordance to previously described protocol (Kutner et al., Nature Protocols, 2009, 4, 495-505).

Math1-CreER mice carrying an inducible Cre recombinase in Math1 expressing cells were crossed with Ptch1^{+/-} mice and R26R-eYFP mice with a loxp-flanked STOP cassette upstream of eYFP gene (Mao et al., Blood, 2001, 97, 324-326, and Machold et al., Neuron, 2005, 48, 17-24). After tumor was established at around 30 weeks, Math1-CreER/Rosa-eYFP/Ptch1^{+/-} mice were treated with tamoxifen by oral gavage. Conditional NSDHL knockout mice, in which a proportion of NSDHL gene was flanked by loxp sites (Cunningham et al., Human Mol. Genet., 2015, 24, 2808-2825), were then crossed with Math1-CreER mice and Ptch1^{+/-} mice. Math1-CreER/NSDHL-loxp/Ptch1 ^{+/-} mice and NSDHL-loxp/Ptch1^{+/-} mice were orally treated with tamoxifen after tumor was formed.

Histology and immunohistochemistry: Frozen sections were prepared according to standard protocol (Yang et al., 2008, Cancer Cell, 14, 135-145). Briefly, tissue was fixed in 4% paraformaldehyde overnight, cryoprotected in 30% sucrose, embedded in O.C.T., and sectioned for 12 μm thick slides. Samples were permeabilized and blocked with 10% normal goat serum on PBST (PBS, 0.1% Triton-X100) and stained against Ki67 (Abcam, ab15580, 1:1000), NeuN (Abcam, ab104224, 1:200), Caspase 3 (Cell Signaling, 9662, 1:500), BrdU (Sigma-Aldrich, B-8434, 1:500), mCherry (Abcam, ab167453, 1:500), Zic1 (Generous gift from Dr. Sigal, Dana Farber Research Institute, 1:200), S10013 (Sigma-Aldrich, 1:500), and YFP (Life Technologies, 1:500).

Cultured primary MB cells were fixed with 4% PFA on PBS for 15 minutes, permeabilized, and blocked in 10% normal goat serum on PBST (PBS, 0.1% Triton-X100) for 1 hour at room temperature. Prior to BrdU immunostaining, cells were incubated in 4.2 mM MgCl₂/0.15 M NaCl pH 5.0 buffer in the presence of 50 U/mL of DNase I (Sigma-Aldrich) at 37° C. for 30 minutes. Secondary antibodies were applied at a dilution of 1:500 on 10% normal goat serum on PBST for 2 hours at room temperature. Samples were counterstained with DAPI and mounted with Fluoromount-G.

Western blotting: Cells were lysed in RIPA buffer (Thermo, 89901) supplemented with protease (Roche, 11836170001) and phosphatase (Thermo, 78428) inhibitors cocktail. Total protein lysates were separated in 8% SDS-PAGE and transferred on PVDF membrane. Membranes were probed with antibodies against Gli1 (Cell Signaling, 2534S, 1:1000), NSDHL (Proteintech, 15111-1-AP, 1:1000) and GAPDH (Sigma-Aldrich, G8795, 1:1000). Western Blot signals were detected with SuperSignal West Pico Substrate and exposed on films. Mass-spectrometry cholesterol assay: Cholesterol concentration in cell or tissue sample was evaluated according previously described protocol (McDonald et al., Methods Enzymol, 2007, 432, 145-170). Briefly, each cell (10⁶ cells) or tissue sample (50 mg) was washed with ice-cold DPBS, and lipids were extracted using a Bligh/Dyer procedure (Bligh and Dyer, Can J Biochem Physol, 1959, 37, 911-917), in which 6 mL of 1:2 (v/v) chloroform:methanol was added to the sample. ²H₆-26,26,26,27,27,27-cholesterol (Sigma-Aldrich, 679046) was used as the internal standard spiked in individual samples. Cholesterol-containing chloroform layer was collected and evaporated under a gentle, dried nitrogen stream at 35° C. The dried samples were re-dissolved in 200 μL of methanol and analyzed by ultra performance liquid chromatography-tandem mass spectrometry (UPLC-MS/MS). The instrumental parameters of MS were set as described previously (Kuo et al., Plos One, 2013, e54896). The peaks of samples were processed and analyzed using Xcalibur, version 2.1.

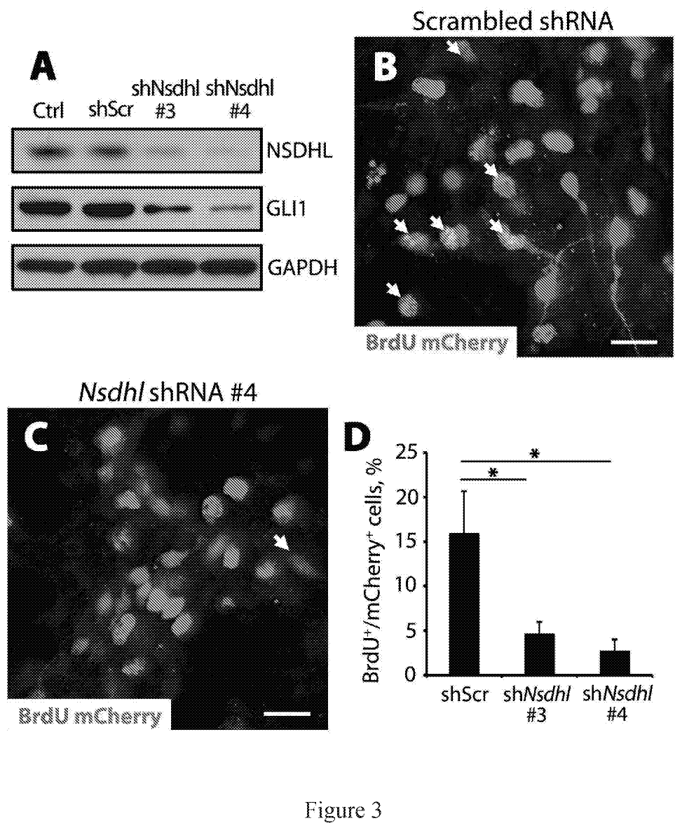

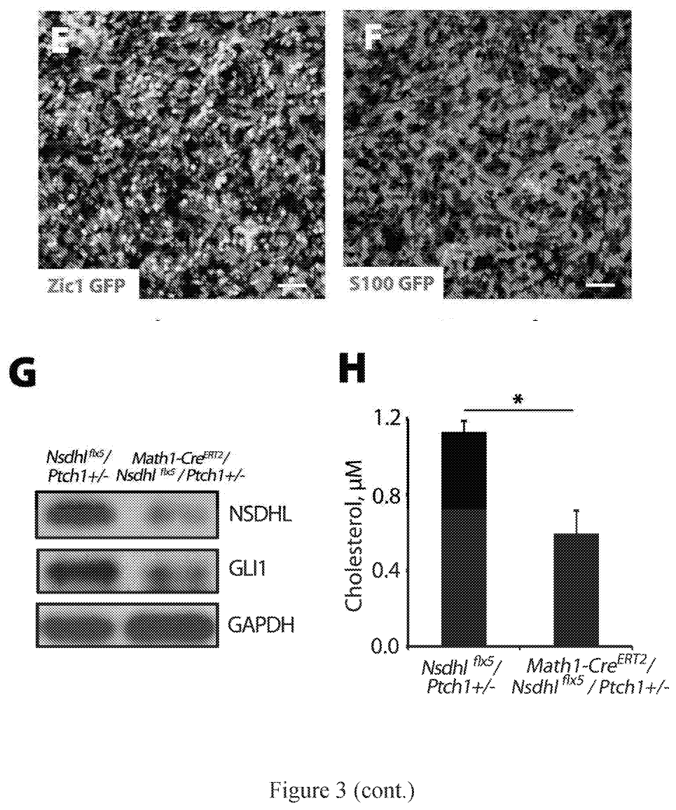

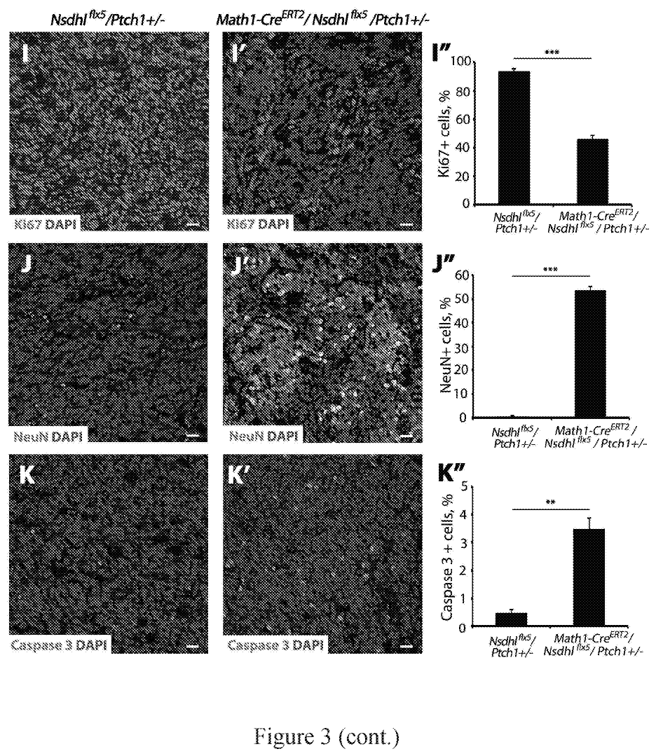

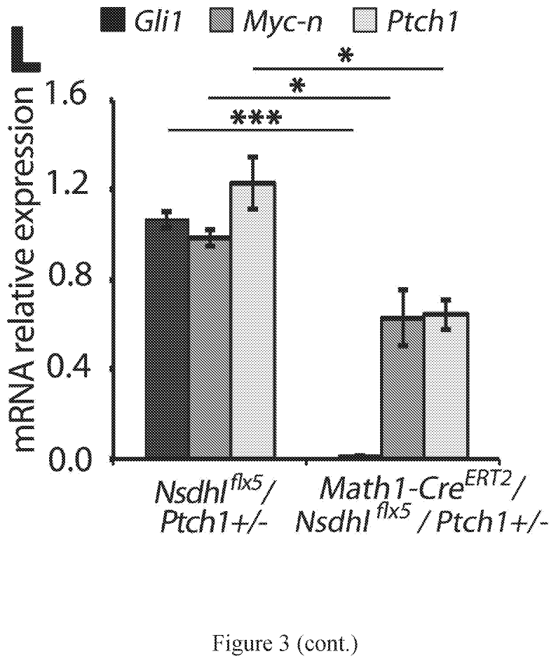

FIG. 3 (panels A-D) shows depletion of NAD(P) dependent steroid dehydrogenase-like (NSDHL) abolished Hh-dependent MB cell proliferation. FIG. 3 (panel A) shows a Western blot analysis of NSDHL and GLI1 proteins expression in Ptc^{+/-} MB cells following infection with lentivirus carrying Nsdhl shRNA (#3 and #4), scrambled shRNA, and uninfected control after 48 hours in culture. GAPDH was used as a loading control. FIG. 3 (panels B and C) shows immunofluorescence analysis of BrdU positive cells among scrambled control shRNA (see, FIG. 3, panel B) and Nsdhl shRNA #4 (see, FIG. 3, panel C) carrying mCherry positive MB cells after 48 hours in culture. FIG. 3 (panel D) shows the percentage of BrdU/mCherry double-positive cells represented on FIG. 3 (panels B and C).

In particular, at 48 hours following virus infection, NSDHL protein expression was effectively repressed in MB cells by NSDHL shRNAs (see, FIG. 3, panel A). Reduced Gli1 expression in NSDHL-deficient MB cells, indicates that NSDHL knockdown impaired Hh signaling activation in MB cells. Moreover, proliferation of MB cells after virus infection was examined by BrdU incorporation assay. As shown in FIG. 3 (panels B-D), around 15% of scramble shRNA-infected MB cells were still dividing (BrdU+), whereas only 4-6% of NSDHL-deficient MB cells were positive for BrdU. These data confirm that cholesterol synthesis is critical for the proliferation and maintenance of Hh signaling in MB cells.

FIG. 3 (panel E) shows Math1-CreERT2/R26R-eGFP/Ptc^{+/-} MB cerebellar section stained against Zic1 and GFP. In particular, FIG. 3 (panel E) demonstrates that most of tumor cells (Zic1+) expressed GFP, and no astrocytes (S100+) or oligodendrocytes (O4+) were positive for GFP (data not shown). These data indicate that Math1-CreER mice could be utilized to target MB cells in vivo.

FIG. 3 (panel F) shows Western blot analysis of NSDHL and GLI1 protein expression in Math1-CreERT2/Nsdhl flx5/Ptc^{+/-} MB and Ptc^{+/-} control tissues after a week of tamoxifen treatments. GAPDH was used as a loading control. FIG. 3 (panel G) shows mass-spectrometry analysis of cholesterol content (μM) in Math1-CreERT2/Nsdhl flx5/Ptc^{+/-} MB and Ptc^{+/-} control tissue. FIG. 3 (panel H) shows qPCR analysis of relative mRNA expression of Hh pathway genes (Gli1, n-Myc, Ptch1) in Math1-CreERT2/Nsdhl flx5/Ptc^{+/-} MB and Ptc^{+/-} control tissue. FIG. 3 (panels I-I″) shows immunofluorescent analysis of Ki67 staining in Ptc^{+/-} (see, FIG. 3, panel I), Math1-CreERT2/Nsdhl flx5/Ptc^{+/-} (see, FIG. 3, panel I′), and percentage of Ki67 positive cells in above sections (see, FIG. 3, panel I″), as well as NeuN (see, FIG. 3, panels J-J″) and cleaved caspase 3 expression (see, FIG. 3, panels K-K″). DAPI was used as a counterstain. Scale bars are 10 μm. Significance asterisk key: p value <0.05 (*), p<0.01 (**), p<0.001 (***), p<0.0001 (****).

As shown in FIG. 3 (panel F), NSDHL gene was largely deleted in MB cells, confirmed by Western blotting. Moreover, the amount of cholesterol in tumor tissue was dramatically reduced after tamoxifen treatment, revealed by MS-Spec analysis (see, FIG. 3, panel G). As shown in FIG. 3 (panels I-I″), tumor cell proliferation was significantly prohibited in NSDHL deficient MB tissue compared with the control, as shown by immunostaining for Ki67. Instead, most of NSDHL-deficient tumor cells underwent differentiation (NeuN+) (see, FIG. 3, panels J-J″). Increased apoptosis (Cleaved caspase-3+) was detected in tumor tissue after NSDHL deletion (see, FIG. 3, panels K-K″). Consistent with these findings in the cultured cells, expression of Hh signaling target genes including Gli1, Myc-N and Ptch1 in MB tissue was significantly repressed after NSDHL deletion, supporting that NSDHL deficiency compromised Hh signaling in MB cells (see, FIG. 3, panel L). The above data support that cholesterol promotes tumor cell proliferation and in vivo growth of MB cells.

### Example 4: Cholesterol Promotes Smo Activation

Having observed that MB cell proliferation was compromised after cholesterol deficiency, it was postulated that cholesterol may promote Hh pathway activation. Western blotting: Cells and tissues were lysed in RIPA buffer (Thermo) supplemented with protease (Roche) and phosphatase (Thermo) inhibitors cocktail. Total protein lysates were separated in 8% SDS-PAGE and transferred on PVDF membrane. Membranes were probed with antibodies against GLI1 (Cell Signaling, 1:1000), NSDHL (Proteintech, 1:1000), Ptch1 (Novus Biologicals, 1:1000), GAPDH (Sigma-Aldrich, 1:1000). Western Blot signals were detected with SuperSignal West Pico Substrate and exposed on films.

MB cells isolated from Ptch1^{+/-} mice were treated with Simvastatin with and without addition of WSC. At 48 hours after the treatment, cells were harvested to measure Hh signaling through examining Gli1 expression by Western blotting. To further examine the requirement of cholesterol in Hh signal transduction, NIH3T3 cells that have all components involved in Hh signaling were utilized.

Primary mouse embryonic fibroblasts (MEFs) from Ptch1fl/fl mice carrying a proportion of Ptch1 gene flanked by loxp sites (Ellis et al., Genesis, 2003, 36, 158-161), or SmoM2 mice expressing a constitutively activated form of Smo upon Cre recombination (Jeong et al., Genes & Develop., 2004, 18, 937-951) were generated. MEFs were then infected with a lentivirus carrying Cre recombinase to activate Hh signaling, or with an empty vector as a control. At 48 hours after the infection, MEFs were harvested to examine Gli1 protein expression by Western blotting.

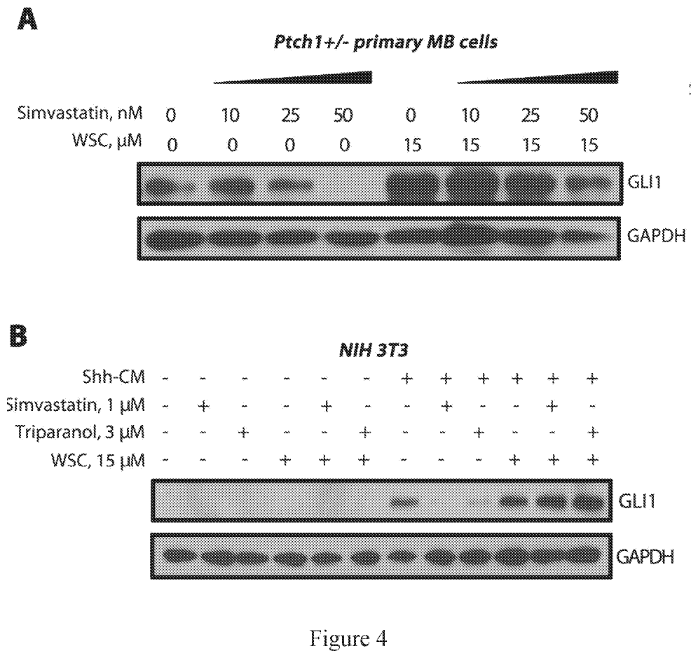

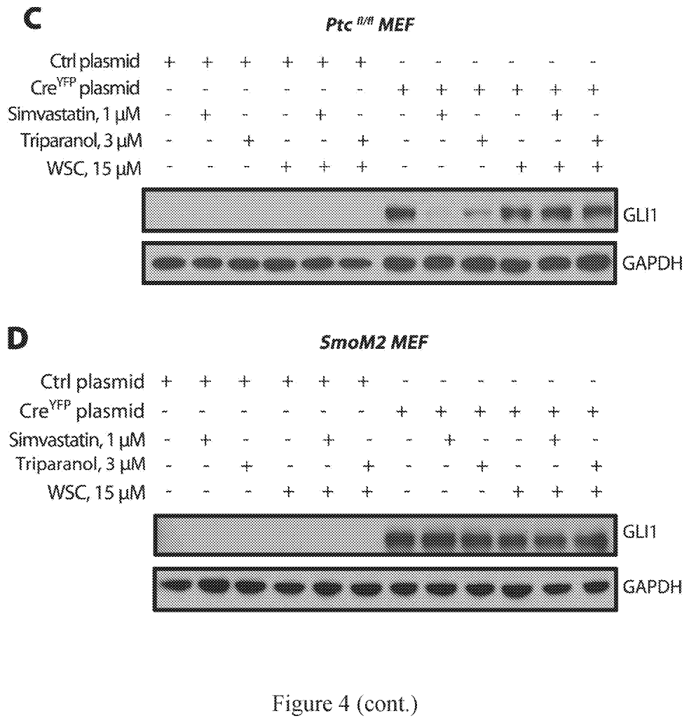

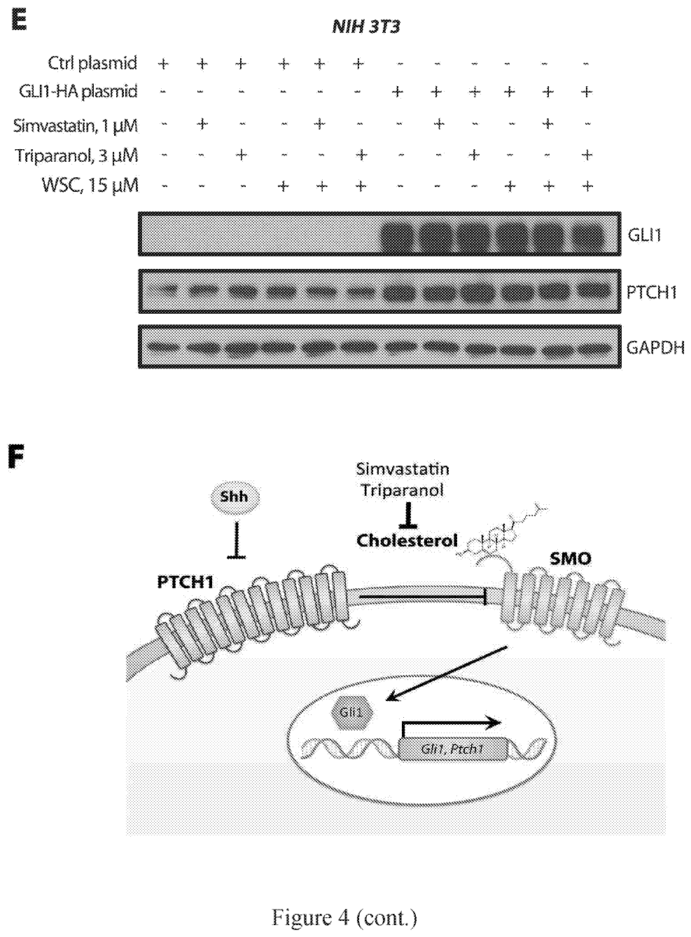

FIG. 4 (panel A) shows cholesterol promoted Smo activation during Hh signal transduction as revealed by Western blot analysis of GLI1 protein expression in Ptc^{+/-} MB cells following simvastatin and water soluble cholesterol treatment after 48 hours in vitro. As shown in FIG. 4 (panel A), the expression levels of Gli1 protein declined as the concentration of simvastatin increased. Moreover, the addition of exogenous cholesterol rescued Gli1 expression in Simvastatin treated MB cells. These data support that cholesterol deficiency after simvastatin treatment impaired Hh signaling in MB cells.

Simvastatin or triparanol significantly repressed Gli1 protein expression in NIH3T3 cells upon Hh treatment. Exogenous cholesterol upregulated Gli1 protein expression in NIH3T3 cells after Simvastatin or Triparanol treatment, supporting that simvastatin or triparanol-inhibited Gli1 expression was indeed due to cholesterol deficiency. The above data confirmed that cholesterol promotes the signal transduction of Hh pathway.

FIG. 4 (panel B) shows Western blot analysis of GLI1 protein expression in NIH3T3 cells following simvastatin, triparanol and water soluble cholesterol treatment for 48 hours in vitro. FIG. 4 (panels C and D) show Western blot analysis of GLI1 protein expression in Ptcfl/fl (see, FIG. 4, panel C) and SmoM2 MEFs (see, FIG. 4, panel D) following CreYFP carrying lentivirus infection and subsequent simvastatin, triparanol and water soluble cholesterol treatment for 48 hours in vitro. FIG. 4 (panel E) shows Western blot analysis of GLI1 and PTCH1 proteins in NIH3T3 cells following Gli1-HA overexpression construct transfection and subsequent simvastatin, triparanol and water soluble cholesterol treatment for 48 hours in vitro. GAPDH was used as a loading control. FIG. 4 (panel F) shows a schematic diagram of Hh signaling pathway. In the absence of Hh ligand, its receptor Ptch1, tethers pathway effector Smo. Bound to Ptch1, Hh releases Smo, causes activation of Gli1 transcription factor, and leads to transcription of Hh pathway genes (Gli1, Ptch1, etc.). Cholesterol promotes Smo activation during Hh signal transduction.

As shown in FIG. 4 (panels C and D), Hh pathway in MEFs was activated following Cre virus infection, evidenced by elevated levels of Gli1 protein expression. Treatment with simvastatin or triparanol dramatically inhibited Gli1 expression in Ptch1-deficient MEFs, and such inhibition was restored by addition of exogenous cholesterol. However, up-regulation of Gli1 protein expression in Smo-activated MEFs was not altered by simvastatin or triparanol treatment. These data support that cholesterol functions at the level of Smo during Hh signaling activation. To further confirm this observation, Hh signaling in NIH3T3 cells was activated by overexpression of Gli1, acting downstream of Smo in Hh signaling. After infection with a lentivirus carrying Gli1 expression vector, or an empty vector as a control, robust expression of Gli1 protein in Gli1-overexpressed NIH3T3 cells. Increased levels of Ptch1 protein indicate the activation of Hh signaling in NIH3T3 cells after Gli1 overexpression (see, FIG. 4, panel E). Upregulation of Ptch1 protein expression in Gli1-overexpressed NIH3T3 cells was not altered by treatment with simvastatin or triparanol. These data indicate that cholesterol promotes Smo activation during Hh pathway signal transduction (see, FIG. 4, panel F).

### Example 5: Antagonists of Cholesterol Synthesis Synergize with Smo Antagonist in MB Treatment

Having observed the important role of cholesterol in MB growth, whether pharmaceutical inhibition of cholesterol synthesis could be utilized to treat MB was examined In addition, the effect of cholesterol inhibition on MB cells derived from Smo activation was further examined The synergistic effect of simvastatin and GDC0449 in treating MB was also examined.

An MB model by subcutaneous injection of MB cells isolated from Ptch1+/− mice into SCID mice (Sasai et al., Cancer Research, 2006, 66, 4215-4222) was generated. After tumor volume reached 200-400 mm³, mice were treated with simvastatin or vehicle as a control (IP injection). Subcutaneous tumors were generated by injection of MB cells developed from constitutively activated Smo (Math1-Cre/SmoM2 mice (Schuller et al., Cancer Cell, 2008, 14, 123-134)). Tumor-bearing mice were treated with oral gavage of vismodegib (GDC0449), simvastatin (IP injection), or in combination.

Subcutaneous allografts: CB17/SCID male mice of age 6-8 weeks were used for subcutaneous allograft establishment. Isolated 2×10⁶ single-cell suspension from tumor-bearing mouse was mixed with Reduced growth factor Matrigel (Corning) and injected subcutaneously on the flank of animal Prior to drug treatment experiment, tumor volume was measured and animals that satisfied initial tumor volume requirement (200-400 mm³) were randomized in experimental groups with equivalent number of animals in each group. Animals in which tumor volume did not meet required criteria were not used for further experiments.

Compounds simvastatin and vismodegib (GDC-0449) were administrated in half of indicated dose twice a day by intraperitoneal injection and per os respectively. Tumor size measurements were taken every 2 days and tumor volume calculated as described previously (Tomayko et al., Cancer chemotherapy and Pharmacology,1989, 24, 148-154).

qPCR: RNA was isolated using TRI reagent (Sigma-Aldrich, T9424) in RNase-free conditions. cDNA was synthesized using oligo(dT) and Superscript II reverse transcriptase (Invitrogen, 18064014). Quantitative PCR reactions were performed in triplicates using iQ SYBR Green Supermix (Bio-Rad) and the Bio-Rad iQ5 Multicolor Real-Time PCR Detection System.

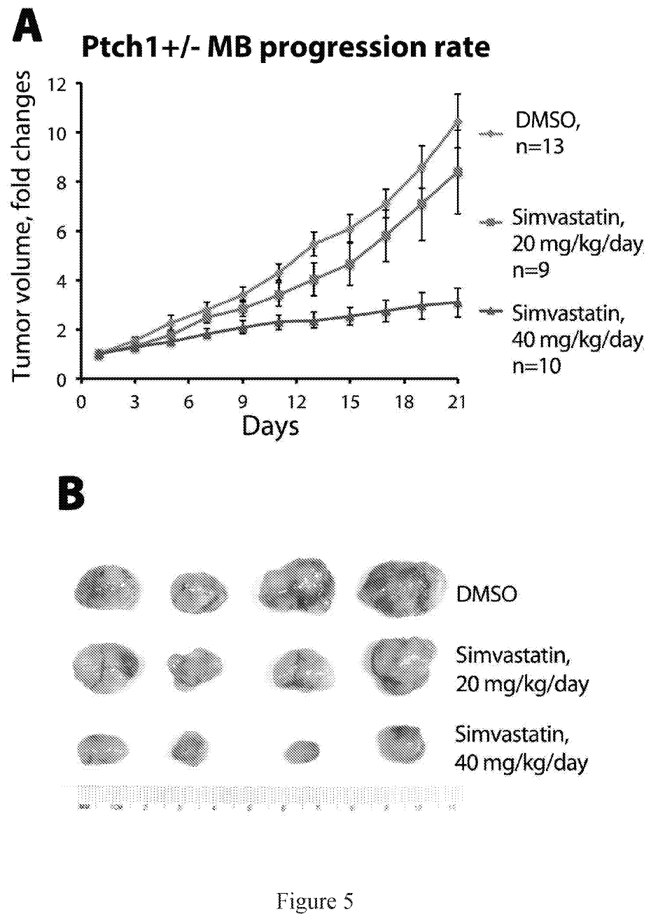

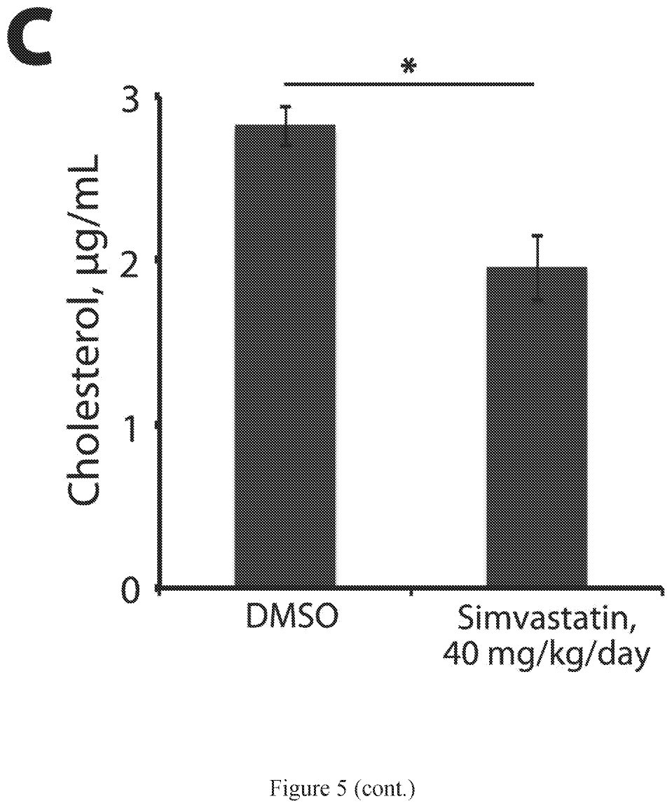

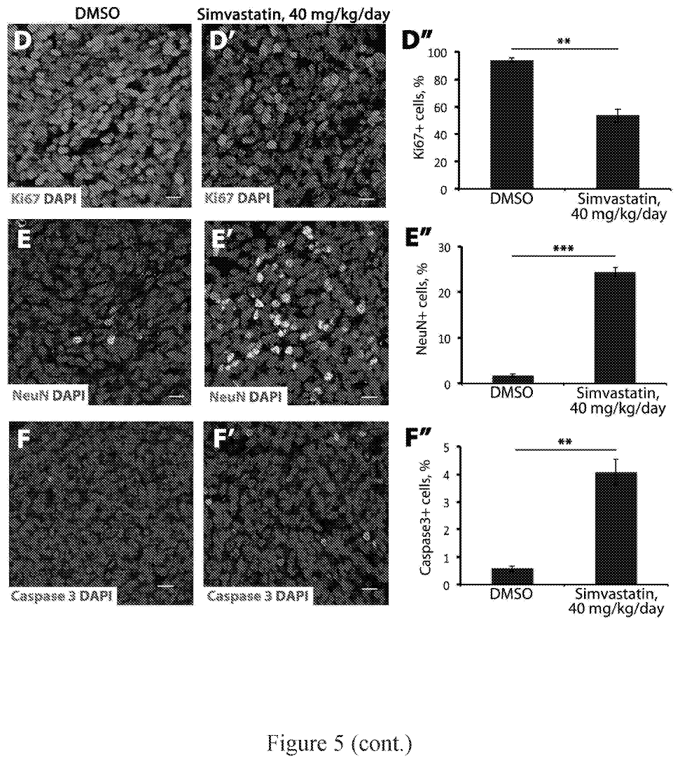

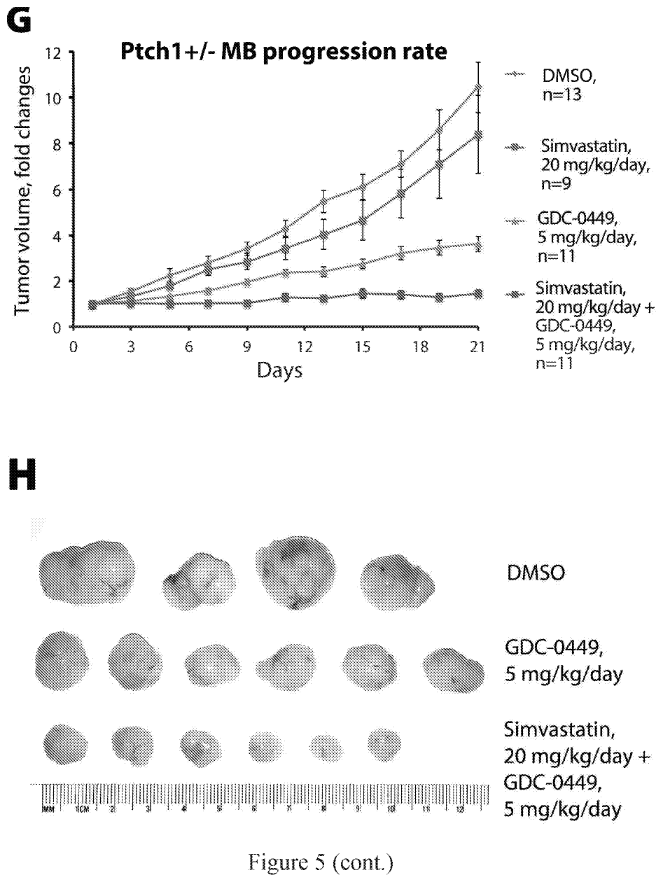

FIG. 5 (panels A-H) show simvastatin blocked MB allograft growth and synergized with Smo antagonist. FIG. 5 (panel A) shows Ptc^{+/-} MB subcutaneous allograft progression rate following simvastatin treatment and control. FIG. 5 (panel B) shows Ptc^{+/-} MB subcutaneous tumors size after 3 weeks of treatment with simvastatin. FIG. 5 (panel C) shows cholesterol content analysis in control and 40 mg/kg/day simvastatin treated subcutaneous tumors after 3 weeks of treatment. FIG. 5, (panels D-D″) show immunofluorescent analysis of Ki67 staining in control Ptc^{+/-} (see, FIG. 5, panel D), 40 mg/kg/day simvastatin treated (see, FIG. 5, panel D′) tumors, and percentage of Ki67 positive cells in above sections (see, FIG. 5, panel D″), as well as NeuN (see, FIG. 5, panels E-E″) and cleaved caspase 3 expression (see, FIG. 5, panels F-F″) DAPI used as a counterstain. FIG. 5 (panel G) shows Ptc^{+/-} MB subcutaneous allograft progression rate following 20 mg/kg/day simvastatin, 5 mg/kg/day vismodegib (GDC0449) and their combination. FIG. 5 (panel H) shows Ptc^{+/-} MB subcutaneous tumors size after 3 weeks of treatment with simvastatin, vismodegib (GDC0449) and their combination. Scale bars are 10 μm. Significance asterisk key: p value <0.05 (*), p<0.01 (**), p<0.001 (***), p<0.0001 (****).

As shown in FIG. 5 (panels A and B), although no obvious effects on tumor growth after treatment with 20 mg/kg/day simvastatin, simvastatin at a dosage of 40 mg/kg/day significantly inhibited MB progression in vivo. Reduced cholesterol levels in tumor tissue after simvastatin treatment was confirmed by mass-spectrometry analysis (see, FIG. 5, panel C). Immunochemical analysis revealed that simvastatin treatment repressed tumor cell proliferation (Ki67+), and promoted differentiation (NeuN+) among MB cells. Increased number of Cleaved Caspase-3 positive cells was found in tumor tissue after simvastatin treatment, indicating that blockage of cholesterol synthesis induced apoptosis in tumor cells (see, FIG. 5, panels D-F′). Gli1 protein expression in tumor tissue was significantly down-regulated after simvastatin treatment, indicative of compromised Hh signaling in MB cells following inhibition of cholesterol synthesis. These data support that antagonists of cholesterol synthesis could efficiently inhibit MB growth. No obvious inhibition of MB growth was observed after simvastatin treatment, which is consistent with our findings that cholesterol regulates Hh signaling at the level of Smo. 5 mg/kg/day of vismodegib (GDC0449) treatment prohibited MB growth. However, combined treatment with vismodegib (GDC0449) and simvastatin resulted in a significant inhibition of MB progression. The synergistic inhibitory effects on MB progression was confirmed by the statistical analysis. These data indicate that repressed cholesterol synthesis enhance the efficacies of vismodegib (GDC0449) in inhibition of MB growth.

Various modifications of the described subject matter, in addition to those described herein, will be apparent to those skilled in the art from the foregoing description. Such modifications are also intended to fall within the scope of the appended claims Each reference (including, but not limited to, journal articles, U.S. and non-U.S. patents, patent application publications, international patent application publications, gene bank accession numbers, and the like) cited in the present application is incorporated herein by reference in its entirety.

1. A pharmaceutical composition comprising:
a cholesterol synthesis inhibitor;
an antagonist of the transmembrane protein Smoothened (Smo); and
a pharmaceutically acceptable carrier.

2. The pharmaceutical composition of claim 1 wherein the cholesterol synthesis inhibitor is a Sonic hedgehog (Hh) antagonist, a PCSK9 inhibitor, a cholesterol absorption inhibitor, or a statin.

3-4. (canceled)

5. The pharmaceutical composition of claim 1 wherein the Smo antagonist is vismodegib, cyclopamine, erismodegib, PF-5274857, GANT61, SANT1, glasdegib, taladegib, sonidegib, IPI-926, PF-04449913, Ly-2940680, MRT-83, GSA-10, tomatidine, SANT-2, or BMS-833923.

6. (canceled)

7. The pharmaceutical composition of claim 1 wherein the ratio of the cholesterol synthesis inhibitor to the Smo antagonist is from about 0.01:1 to about 100:1 w/w.

8. The pharmaceutical composition of claim 1 wherein the cholesterol synthesis inhibitor is present in amount from about 5 mg to about 50 mg, and the Smo antagonist is present in an amount from about 25 mg to about 300 mg.

9-11. (canceled)

12. A method of reducing the proliferation of an Hh-associated tumor cell or treating an Hh-associated cancer in a human in need thereof comprising administering to the human a cholesterol synthesis inhibitor.

13. The method of claim 12 wherein the cholesterol synthesis inhibitor is an Hh antagonist.

14. The method of claim 13 wherein the cholesterol synthesis inhibitor is a statin.

15-19. (canceled)

20. The method of claim 12 further comprising administering an antagonist of the transmembrane protein Smoothened (Smo) to the human.

21. The method of claim 20 wherein the Smo antagonist is vismodegib, cyclopamine, erismodegib, PF-5274857, GANT61, SANT1, glasdegib, taladegib, sonidegib, IPI-926, PF-04449913, Ly-2940680, MRT-83, GSA-10, tomatidine, SANT-2, or BMS-833923.

22-23. (canceled)

24. The method of claim 20 wherein the cholesterol synthesis inhibitor is administered prior to the administration of the Smo antagonist or after the administration of the Smo antagonist.

25. The method of claim 20 wherein the cholesterol synthesis inhibitor is administered concurrently with administration of the Smo antagonist.

26. The method of claim 20 wherein the cholesterol synthesis inhibitor and the Smo antagonist are present in the same pharmaceutical composition.

27. The method of claim 26 wherein the cholesterol synthesis inhibitor is present in the pharmaceutical composition in an amount from about 5 mg to about 50 mg, and the Smo antagonist is present in the pharmaceutical composition in an amount from about 25 mg to about 300 mg.

28-30. (canceled)

31. The method of claim 12 wherein the human is also administered radiation therapy and/or chemotherapy.

32. The method of claim 12 wherein the Hh-associated cancer is breast cancer, stomach cancer, liver cancer, prostate cancer, small-cell lung cancer, pancreatic cancer (PDAC), colon cancer, esophagus cancer, biliary tract cancer, medulloblastoma, basal cell carcinoma, or rhabdomyosarcoma.

33-36. (canceled)

37. A pharmaceutical composition comprising a cholesterol synthesis inhibitor for use in the manufacture of a medicament for reducing the proliferation of an Hh-associated tumor cell or treating an Hh-associated cancer in a human.

38-40. (canceled)

41. A method of culturing medulloblastoma cells comprising:
contacting the medulloblastoma cells with growth media; and
contacting the medulloblastoma cells with an amount of cholesterol that is sufficient to maintain the cells in culture for up to 96 hours at least.

42. The method of claim 41 wherein the growth media is NB-B27.

43-45. (canceled)

46. A kit comprising:
growth media for culturing medulloblastoma cells; and
cholesterol.
